(19) United States
(12) Patent Application Publication
(10) Pub. No.: US 2004/0193127 A1
(43) Pub. Date: Sep. 30, 2004

(54) Absorbent article comprising an absorbent structure

(75) Inventors: Charlotta Hansson, Goteborg (SE); Ted Guidotti, Goteborg (SE); Eje Osterdahi, Vastra Frolunda (SE); Anna Bagger-Sjoback, Goteborg (SE)

Correspondence Address: BURNS DOANE SWECKER & MATHIS L L P, POST OFFICE BOX 1404, ALEXANDRIA, VA, 22313-1404, US

(73) Assignee: SCA Hygiene Products AB

(21) Appl. No.: 10/809,642

(22) Filed: Mar. 25, 2004

Related U.S. Application Data
(60) Provisional application No. 60/457,313, filed on Mar. 26, 2003.

(51) Int. Cl. A61F 13/15; A61F 13/20

(52) U.S. Cl. 604/367; 604/385.01

(57) ABSTRACT

An absorbent article such as a diaper, an incontinence guard, a sanitary napkin or the like, exhibits a liquid permeable surface and an opposite at least substantially liquid impermeable surface and has an absorbent structure. The absorbent structure has a super absorbent porous structure exhibiting a Gurley stiffness value being lower than 1000 mg and a density in a dry condition exceeding 0.5 g/cm³.

This application is related to, and claims priority from, U.S. Provisional Application Serial No. 60/457,313 filed on Mar. 26, 2003, the disclosure of which is expressly incorporated herein by reference.

### FIELD OF THE INVENTION

The present invention relates to an absorbent article such as a diaper, an incontinence guard, a sanitary napkin or the like, whereby the article exhibits a liquid permeable upper surface and an opposite at least substantially liquid impermeable surface and comprising an absorbent structure.

### BACKGROUND ART

Absorbent structures for absorbent articles such as diapers, incontinence guards, sanitary napkins, intended for one single use, are usually constructed by one or more layers of hydrophilic fibers, for example cellulosic fluff pulp. Frequently, a super absorbent material is comprised in such structure, said superabsorbent materials being polymers having the ability to absorb water or bodily fluids many times their own weight. Further, the absorbent structure may comprise other components, for example to improve its liquid distribution capacity or increase its ability to keep the structure together and ability to resist deformation during use.

A huge problem, mainly for diapers and incontinence guards for adults intended to receive and absorb relatively large amounts of liquid, is that they leak before their total absorption capacity has been fully utilized. Since upon urination often large amounts of liquid are discharged during the course of a few seconds, this may lead to that the absorbent structure locally is saturated with liquid in the liquid acquisition zone, i.e., the liquid will not be distributed to the other portions of the absorbent structure in a sufficiently short period of time. This will lead to that further liquid discharged by the user will leak out from the diaper. Naturally, such premature leakage can be very annoying, both for the user and for the nursing personnel. The leakage problem is accentuated upon repeated wetting.

In EP 0 254 476 an absorbent structure is described, in which one zone is being arranged substantially just before the wetting area, exhibiting a lower grammage and a lower density compared to surrounding portions of the absorbent structure. Consequently, a high momentary liquid absorption capacity in the wetting area is obtained whereby the liquid can be further distributed and stored in surrounding portions of the absorbent structure.

In GB 2 082 643 an absorbent structure is described having a substantially equal grammage but exhibits a density gradient in the longitudinal direction, so that the density increases toward the end portions of the absorbent structure.

In U.S. Pat. No. 4,413,996 an absorbent structure for diapers is described exhibiting a liquid absorbent depression or well in the wetting area. In this well a porous pad can be arranged.

However, there is still a need for improvements regarding liquid absorption capacity and distribution capacity, mainly for products intended to receive large amounts of liquids during a short period of time.

### SUMMARY

According to one or more embodiments of the present invention, an article has been provided, which is thin and discrete to wear, and at the same time the article exhibits sufficient liquid acquiring capacity, liquid distribution capacity and liquid storage capacity.

An absorbent article according to an embodiment of the invention is characterized mainly in that the absorbent structure comprises a super absorbent porous structure exhibiting a Gurley stiffness value being lower than 1000 mg and a density in a dry condition exceeding 0.5 g/cm³.

The dry density value relates to the density the super absorbent porous structure exhibits in the absorbent article upon storage of absorbent articles in a sealed diaper package. The term “diaper package” denotes the package, in which the diapers are enclosed when selling the diapers. In some cases, the diapers are packed one by one, whereby a number of single-wrapped diapers then are further enclosed in a bigger package. Therefore, the term “diaper package” does not denote a single-wrapped diaper but the bigger diaper package. Analogously, when the absorbent article is an incontinence guard or a sanitary napkin, the density of the structure is referred to as the exhibited density in the absorbent structure comprised in said articles within a sealed package for incontinence guards or sanitary napkins. Measurements of the density of the fragments must be made within 2 minutes after opening the package in question. The density should be measured at a load against the material being 0.5 kPa.

If the super absorbent porous structure is adhesively joined or in another way fastened to any layer upon usage of the absorbent material in the article, the density is first measured on the super absorbent porous structure and the material being attached to the super absorbent porous structure. This measurement is performed within 2 minutes from the opening of the package. Thereafter the attached material is removed from the super absorbent porous structure, whereby the density is measured on the attached material. Thus, the density of the super absorbent porous structure can be calculated.

A super absorbent foam material according to an embodiment of the invention is at least upon storage in the diaper package thin and compressed. According to a preferred embodiment, such a super absorbent porous structure is also thin and compressed upon usage of the absorbent article comprising the super absorbent porous structure. According to a preferred embodiment, such a super absorbent porous structure also upon usage has a density in a dry condition exceeding 0.5 g/cm³. Such a super absorbent foam material expands heavily upon contact with water. Upon the expansion, the free volume of the material is increased, leading to that such a super absorbent material can receive a large amount of liquid during a short period of time. Preferably, the super absorbent porous structure constitutes the acquisition portion comprised in the absorbent structure. The term acquisition portion denotes firstly, the layer receiving the liquid (i.e., the acquisition layer) and secondly, the layer absorbing the liquid from a first inlet layer and then intermediary store the liquid (i.e., an intermediate storage layer) before the liquid eventually is being stored in the final storage portion.

According to a preferred embodiment, the super absorbent porous structure is a polyacrylate-based foam material. A polyacrylate-based foam material is produced by the saturation under pressure using carbon dioxide of a solution, which at least contains monomer, a cross-linking material, an initiator and a tenside in a vessel during stirring. When the solution is removed from the vessel through a nozzle, the solution is expanded and a foamed structure is achieved. The foamed structure is then locked in that polymerization and cross-linking are initiated by for instance UV radiation and/or e-beam. Finally, the material is compressed and dried.

According to a preferred embodiment, the super absorbent porous structure exhibits a density in a dry condition exceeding 0.7 g/cm³. The dry density value relates to the density exhibited by the super absorbent porous structure in the absorbent article upon storage of absorbent articles in a sealed diaper package. The term “diaper package” denotes the package, in which the diapers are enclosed when selling the diapers. In some cases, the diapers are packed one by one, whereby a number of single-wrapped diapers then are further enclosed in a bigger package. Therefore, the term “diaper package” does not denote a single-wrapped diaper but the bigger diaper package. Analogously, when the absorbent article is an incontinence guard or a sanitary napkin, the density of the structure is referred to as the exhibited density in the absorbent structure comprised in said articles within a sealed package for incontinence guards or sanitary napkins. Measurements of the density of the fragments must be made within 2 minutes after opening the package in question. The density should be measured at a load against the material being 0.5 kPa.

According to another embodiment, the super absorbent porous structure exhibits a Gurley stiffness value being lower than 700 mg. According to yet another embodiment, the super absorbent porous structure exhibits a Gurley stiffness value being lower than 500 mg.

According to an embodiment, the total absorption capacity per volume unit of the super absorbent porous structure in a dry condition is at least 15 g/cm³. Thus, we have measured the total absorption capacity in gram liquid per cubic centimeter of the super absorbent porous structure in a dry condition. According to more preferred embodiment, the total absorption capacity per cubic centimeter of the super absorbent porous structure in a dry condition is at least 27 g/cm³. According to yet more preferred embodiment, the total absorption capacity per cubic centimeter of the super absorbent porous structure in a dry condition is at least 35 g/cm³.

Another property of the absorbent structure is that the liquid transport relationship between the acquiring portion and the final storage portion is such that the final storage portion drains liquid from the acquiring portion. Thus, one or more embodiments of the invention also relate to an absorbent structure comprising an acquisition portion and a final storage portion, whereby the super absorbent porous structure constitutes the acquisition portion. The acquisition portion exhibits a drainage rate, measured by a Liquid porosimeter device (from Textile Research Institute, Princeton, USA), being such that at least 50% of the drainable pores in the super absorbent porous structure are emptied from liquid at a pressure being lower than 12 cm H₂O.

According to a similar embodiment wherein the absorbent structure comprises an acquisition portion and a final storage portion, the acquisition portion exhibits a drainage rate, measured by a Liquid porosimeter device, being such that at least 50% of the drainable pores in the super absorbent porous structure are emptied from liquid at a pressure being lower than 8 cm H₂O. According to a more preferred embodiment, the acquisition portion exhibits a drainage rate, being such that at least 50% of the drainable pores in the super absorbent porous structure are emptied from liquid at a pressure being lower than 6 cm H₂O. However, in order for the material to keep the liquid in satisfactory way, the drainage rate of the acquisition portion should not be lower than 2 cm H₂O, and more preferred not lower than 4 cm H₂O. In example 2 the method for the measurement of the drainage rate is described in more detail.

According to one embodiment, the final storage portion at least comprises a first storage layer, wherein the first storage layer is comprised of cellulosic fibers and super absorbent material, wherein the amount of super absorbent material calculated on the total weight of the first storage layer in a dry condition is at least 50 percent by weight. According to a similar embodiment, the amount of super absorbent material calculated on the total weight of the first storage layer in a dry condition is at least 70 percent by weight. In order to obtain a very thin absorbent structure, at least in a dry condition, it has been shown to be an advantage to keep the first storage layer highly compressed, for example to a density exceeding 0.4 g/cm³. In order to achieve a rapid inlet of the liquid, it has been shown advantageous that the first storage layer exhibits apertures/recesses.

According to a further embodiment, the absorbent structure also comprises a second storage layer. The second storage layer preferably contains a lower amount of super absorbent material calculated on the total weight of the second storage layer. The second storage layer lies for instance close against the liquid permeable back sheet. Further, the second storage layer has preferably a larger extension than the first storage layer in the plane of the article. Thus, the second storage layer functions as an extra security zone, i.e., it absorbs liquid that might be present outside the first storage layer or outside the acquisition portion.

### BRIEF DESCRIPTION OF THE DRAWINGS

FIG. 1 shows a planar view of an embodiment of an absorbent article according to the invention.

FIG. 2 shows a cross-section of the absorbent article shown in FIG. 1.

### DETAILED DESCRIPTION OF THE PREFERED EMBODIMENTS

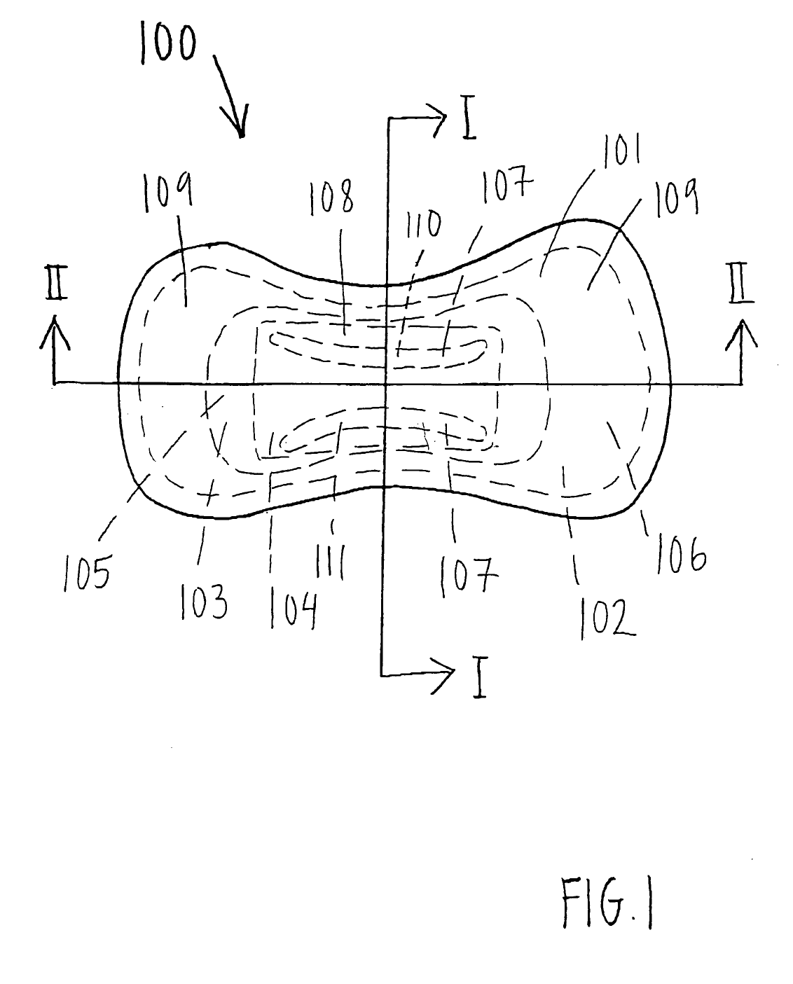

The following description refers only to a couple of embodiments of absorbent articles according to the present invention, which is thus not limited to the below described embodiments. In FIG. 1 a planar view of an absorbent article 100 according to an embodiment of the invention is shown. The absorbent article 100 exhibits a transversal direction, being shown by a transversally extending center line I, and a longitudinal direction being shown by a longitudinally extending center line II. Further, the absorbent article 100 exhibits a thickness direction, being perpendicular to the plane. The absorbent article 100 has a liquid permeable top sheet 101, which during use of the article is intended to lie closest to the user. Further, the absorbent article 100 has a back sheet 102, which is at least substantially liquid impermeable, and an absorbent structure 103 enclosed between the liquid permeable top sheet and the back sheet. The back sheet material 102 can optionally be a so called vapor permeable breathable material. The absorbent structure 103 exhibits a crotch portion 108 and two end portions 109. The absorbent structure 103 comprises an acquisition layer 104, which is intended to rapidly be able receive a large amount of liquid and a first storage layer 105, which is intended to rapidly be able to store a large amount of liquid, and a second storage layer 106. The first storage layer is arranged closest against the liquid permeable top sheet 101, the second storage layer 106 is arranged closest to the substantially liquid impermeable back sheet 102 and the acquisition layer is arranged between the first storage layer 105 and the second storage layer 106. The second storage layer 106 has a longer extension in the plane of the article than the first storage layer 105, but exhibits a lower total absorption capacity. The second storage layer also functions as a form rendering element in such way that it assists in creating and maintaining an absorbent structure being flexible against the body. The first storage layer 105 exhibits two longitudinally arranged apertures/recesses 110, 111. The hollow spaces obtained through the apertures/recesses 110, 111 extend in the longitudinal direction of the article. The distance between the apertures/recesses is preferably maximally 20 mm in the crotch portion. The distance refers to the length of the material between the apertures/recesses 110, 111 in the transversal direction of the article. Close to the end portions 109, the distance between the apertures/recesses is preferably somewhat longer than in the crotch portion. Such a shape is advantageous since both a narrow crotch portion and a shape adapted to the body is obtained.

It is also possible that the apertures/recesses 110, 111 in the first storage layer 105 have other shapes. For example is it possible that the apertures/recesses extend in the transversal direction of the article, whereby transversally arranged channels be obtained. An advantage using this design is that such a storage layer relatively easily may assume a cup shape. Another alternative embodiment is circular apertures/recesses. Naturally, also other shapes of the apertures/recesses are possible.

The storage layers 105, 106 may comprise optional absorbent materials, such as fibrous materials, foam materials, superabsorbent polymers and combinations thereof. According to one embodiment the first storage layer 105 is a fibrous structure and comprises at least 50 percent by weight of a super absorbent material calculated on the total weight of the first storage layer 105. Super absorbent materials are polymers having the capability to absorb water or bodily fluids many times their own weight. Conventionally super absorbent materials are polymers, such as polymers from polyacrylic acid. The super absorbent material is present in the form of powder, flakes, fibers, granules or the like. The super absorbent material in the storage layers 105, 106 may be mixed with the fiber material or may be applied as one or more layers between fiber layers. The super absorbent material is either equally distributed in the first storage layer 105 or distributed in various concentrations in the longitudinal and/or the thickness direction of the first storage layer 105. It is also possible to use a substantially pure layer of super absorbent material in the first storage layer. One example of a suitable super absorbent material is a super absorbent material having the ability to rapidly absorb liquid. A super absorbent material, which can absorb 5 grams of bodily fluids per gram super absorbent material in 10 seconds, is usually defined as a fast super absorbent material. An example of a fast liquid-absorbing super absorbent material is a particulate super absorbent material having a small particle size, i.e., a low particle diameter. Such a particulate super absorbent material usually exhibits an average particle size of between 150 μm and 400 μm. It is also possible to use several types of super absorbent material, for example it is possible to use a super absorbent material which absorbs very rapidly in the first storage layer and a conventional super absorbent material absorbing slower in a second storage layer.

The percentage super absorbent material in the second storage layer 106 is lower than the amount of super absorbent material in the first storage layer 105. For example, the second storage layer 106 may comprise 10 percent by weight of a super absorbent material calculated on the total weight of the second storage layer 106. The second storage layer 106 has a longer extension in the plane of the article, but exhibits a lower total absorption capacity.

The bodily fluids, for example urine, penetrates through the liquid permeable top sheet and is then brought via the hollow spaces, i.e., the apertures/recesses in the first storage layer 105, further to the acquisition layer 104. The acquisition layer 104 is then drained on liquid by the first storage layer 105. The acquisition layer may therefore without difficulties receive a second dose of liquid. The first storage layer has the capacity to store several doses of liquid. Since the first storage layer is heavily compressed, it has been shown difficult for the liquid to penetrate the upper part of the structure. A cellulosic fluff pulp mixed with a high content of super absorbent material, whose structure is highly compressed, exhibits a relatively lustry, glossy upper surface, which is difficult to penetrate for the discharged bodily fluid. By providing the apertures/recesses in the highly compressed material, an access to the inner porous structure is created in the first storage layer. This facilitates the liquid absorbing capacity of the first storage layer.

The acquisition layer 104 is preferably a super absorbent porous structure exhibiting a Gurley stiffness value being lower than 1000 mg and a density in a dry condition exceeding 0.5 g/cm³. Preferably, the super absorbent porous structure is a polyacrylate-based foam material. Examples of a suitable polyacrylate-based foam material are described in detail in Example 2 in the application.

The liquid permeable top sheet 101 may be a nonwoven material or an apertured plastic film, or a laminate thereof. Examples of polymers of which the liquid permeable top sheet can be made of is polyethylene, polypropylene, polyester, or copolymers thereof. To enable the liquid permeable top sheet 101 to rapidly let the discharged bodily fluid through, the top sheet is often coated with tensides and/or is apertured. Since the first storage layer 105 is highly compressed and exhibits a high density, the discharged liquid preferably rapidly reaches the hollow spaces in the first storage layer 105. Therefore, an open liquid permeable top sheet has been shown to be advantageous. An example of an open material is a layer of continuous fibers, which is joined as points, beads or spots, in a binding pattern but are otherwise substantially not connected to each other.

The back sheet 102 can comprise of a plastic layer, a nonwoven layer or a laminate thereof. Preferably, the back sheet 102 is of the breathable kind. All materials being used as back sheet for absorbent articles can be used.

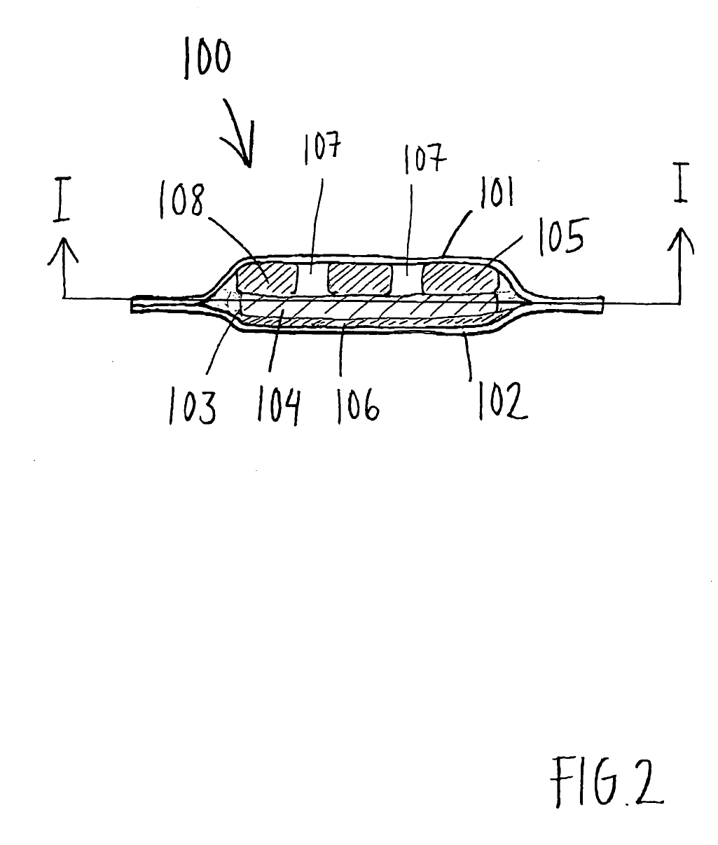

FIG. 2 shows a cross-section through the crotch portion 108 of the absorbent article 100 shown in FIG. 1. Thus, the absorbent article 100 has a liquid permeable top sheet 101, which during use of the article is intended to lie closest to the user, a substantially liquid impermeable back sheet 102, and an absorbent structure 103 enclosed therebetween. The absorbent structure 103 comprises an acquisition layer 104, which is intended to rapidly be able to receive a large amount of liquid, a first storage layer 105, which is intended to rapidly be able to store a large amount of liquid, and a second storage layer 106 having a longer extension in the plane of the article, but exhibits a lower total absorption capacity. The first storage layer 105 exhibits two longitudinally arranged apertures/recesses/hollow spaces 107. The distance of the material between the two apertures/recesses is preferably maximally 20 mm in the crotch portion 108. The first storage layer 105 is arranged closest towards the liquid permeable top sheet 101, the second storage layer 106 is arranged closest to the substantially liquid impermeable backsheet 102 and the acquisition layer 104 is arranged between the first storage layer 105 and the second storage layer 106.

Naturally, the described absorbent article only constitutes an example of an absorbent article according to an embodiment of the invention. In the absorbent article 100 the super absorbent porous structure according to the invention constitutes an acquisition layer, which acquisition layer is arranged between a first storage layer and a second storage layer. It is of course possible that the super absorbent porous structure is an acquisition layer, arranged directly under a liquid permeable top sheet. Further, it is also possible that the super absorbent porous structure is an acquisition layer, arranged closest to the user. In such embodiment, the acquisition layer also functions as a liquid permeable cover layer.

### EXAMPLE 1

Gurley Stiffness

The stiffness of the acquisition layer has been measured. The equipment used for the measurement is the “Gurley Precision Instruments”, made in Troy, N.Y., USA. The measurements were performed according to the method description “Instructions for Gurley bending resistance/stiffness testers, models 4171 C, D, E”. During the measurements, the digital method 4171 D was used.

Tested Materials

Sample 1 is a mixed structure of chemically manufactured cellulosic fluff pulp from Weyerhauser and a particulate polyacrylate-based super absorbent material from BASF. The mixed structure contains 40 percent by weight of super absorbent material based on the total weight of the sample.

Sample 2 is a fiber structure from Weyerhauser. The fiber structure contains 80 percent by weight cross-linked cellulose and 20 percent by weight of thermoplastic fibers.

Sample 3 is a polyester fiber layer having particulate polyacrylate-based super absorbent material bound to the polyester fiber layer. The percentage of super absorbent particles is 60 percent based on the total weight of the sample.

Sample 4 is a polyacrylate-based super absorbent foam layer. The foam layer is denoted Foam XII and is more closely described in Example 2.

Sample 5 is a polyacrylate-based super absorbent foam layer. The foam layer is denoted Foam XV and is more closely described in Example 2.

Sample 6 is a viscose foam material, i.e., a foam material from regenerated cellulose.

The measurements were performed at three different densities for all samples. The samples were measured at the density (ρ) being 0.50 g/cm³, 0.71 g/cm³, and 0.91 g/cm³. The samples were compressed to the given density, i.e., to the density the material exhibits in the package, which density is measured within 2 minutes after the package has been opened. When the samples have been compressed to the given density, the samples were placed in the test equipment after 10-30 seconds. In the table below the results from the measurements are shown. The density is given in g/cm³ and the stiffness in milligrams. The density was measured at a load being 0.5 kPa.

TABLE 1

| | Gurley stiffness (milligrams) | | |
|---|---|---|---|
| | ρ = 0.50 | ρ = 0.71 | ρ = 0.91 |
| Sample | g/cm³ | g/cm³ | g/cm³ |
| Sample 1 | 1343 | 2364 | 2359 |
| Sample 2 | 3437 | 3623 | 3823 |
| Sample 3 | 5090 | 5894 | 6088 |
| Sample 4 | 105 | 69 | 56 |
| Sample 5 | 274 | 123 | 141 |
| Sample 6 | 9246 | 5690 | 5023 |

The results clearly show that the super absorbent foam layers, i.e., sample 4 and sample 5, are significantly softer and more flexible than the other test samples.

### EXAMPLE 2

The drainage rate for polyacrylate-based foam materials have been characterized by means of a Liquid Porosimeter device from Textile Research Institute, Princeton, USA. The function of the equipment is described in detail in Miller, B. and Tyomkin, I. in Journal of Colloid and Interface Science, 162, 163-170 (1994).

The tested materials are two kinds of polyacrylate-based foam materials, Foam XII and Foam XV, respectively. Foam XII has been made according to the following:

To a beaker the following is added:

348.5 grams of acrylic acid (4.84 moles)

135.5 grams of a sodium acrylate solution containing 37.3 percent per weight (0.54 moles)

28.0 grams of polyethylene glycol diacrylate from polyethylene glycol having a molecular weight of 400.

21.3 grams of a aqueous solution 15 percent per weight containing ethylene oxide and linear C₁₆-C₁₈ fatty alcohol (molar ratio 80:1)

65.7 grams of water.

The ingredients were mixed and thereafter, the solution was cooled to a temperature lower than 16° C. The solution was the poured into a closed container, whereby the solution was saturated with carbon dioxide at a pressure of 12 bars for 25 minutes. Using the same pressure, 26.7 grams of an aqueous solution containing 3 percent by weight of 2,2′-azobis(2-amidinopropane) dihydrochloride was added. This was mixed to a homogenous solution. The solution was then allowed to rest in five minutes. The saturated solution was compressed from a container using a nozzle having an opening being 1 mm at a pressure being 12 bars. The resulting monomeric foam was placed on a glass plate (DIN-A3). An additional glass plate was then placed on top of the monomeric foam. Then, the foam was polymerized using a UV/VIS lamp, a UV1000 lamp from Höhnle. The foam was illuminated using the lamp both from underneath and from above. The illumination and thereby also the polymerization were allowed to proceed for 4 minutes. Foam XV was made in the same way. The difference between Foam XII and Foam XV was that more cross-linking agent (i.e., polyethylene glycol diacrylate) was used for making Foam XV. 40.0 grams of polyethylene glycol diacrylate instead of 28.0 grams was added for making Foam XV.

PV₅₀ is the pressure when 50% of the drainable pores have been emptied. The super absorbent foam materials that are shown to be especially advantageous, exhibit a PV₅₀ value being lower than 12 cm H₂O. The PV₅₀ value is obtained by measuring the amount of liquid as a function of the pressure in the chamber in a receding measurement and register when 50% of the drainable pores have been emptied. Upon a receding measurement, the amount of liquid is measured, being emptied from a porous material at a certain pressure in the chamber. At the measurement excess liquid is delivered to the sample. The sample is allowed to absorb this liquid. Then the sample is placed in the chamber on a membrane and a porous plate. A mechanical load is applied. Thereafter the chamber is closed and the air pressure inside the chamber is raised successively in steps by means of a computer-controlled pressure maintaining system, whereby the liquid leaves the sample through a membrane having small pores. The weight of the squeezed liquid is registered using a beam balance.

The amount of liquid present in the sample upon full saturation when it is in the chamber is estimated, Mₒ(g). The liquid remaining in the sample when the pressure in the chamber exceeds 50 cm H₂ is considered to be a difficultly drainable liquid. This amount of liquid is estimated M_{D}(g). The pressure at which 50% of the drainable liquid has been drained out of the samples is calculated according to the following:

M_{50%}=0.5(Mₒ−M_{D})+M_{D}

From the protocol from receding measurements, the pressure at which the chamber had when the sample contained the amount M_{50%}, can be estimated. This pressure is the PV₅₀ value. PV₅₀ should be lower than 12 cm H₂O, more preferably lower than 8 cm H₂O, and most preferred lower than 6 cm H₂O. For the material to be able to keep the liquid in a satisfactory manner, the PV₅₀ value should not be lower than 2 cm H₂O, and preferably not lower than 4 cm H₂O.

A more detailed description of how the measurements were performed follows below.

Before the measurement the samples were kept in sealed plastic bags in order to avoid absorption of moisture from the air. The dry sample was weighed. Thereafter, the sample was placed in the test chamber on membranes (Millipore 0.22 μm cat. No. GSWP 09000), whereby the sample was allowed to swell in excess liquid during 30 minutes. The size of the sample after swelling was 10-25 cm².

At this measurement synthetic urine was used. The ion concentration in the liquid was 0.135 M sodium, 0.086 M potassium, 0.0035 M magnesium, 0.002 M calcium, 0.19 M chloride, 0.0055 M sulphate, and 0.031 phosphate. Additionally, the liquid contained 0.3 M urea and 1 ppm w/w Triton TX-100 (Calbiochem-648462). The liquid was made in such a way that no salts were precipitated.

A load covering the whole sample surface was placed on the sample during the swelling and the measurement. To avoid measuring pores between the sample surface and the load and to maintain an equal load distribution over the whole sample surface, non-absorbent polyurethane foam was placed between the sample and the applied load. The total load put on the swelled sample was 0.3 kPa.

The equilibrium velocity, i.e., the velocity when the weight change at the selected air pressure had decreased to an insignificant level, was upon measuring 5 mg/min and the measure time during which the weight change was recorded was 30 seconds. The measurements were made at the following applied air pressures measured in cm H₂O: 1.1-1.2-1.4-1.7-2.0-2.2-2.4-2.7-3.1-3.5-4.1-4.4-4.7-5.1-5.6-6.1-6.8-7.7-8.7-10.2-11.1-12.2-13.6-15.3-17.5-20.4-24.5-30.6-40.8-49.0-61.2.

To record the remaining liquid, the sample was weighed directly after each terminated measurement. In addition to the measurement on samples one blank control run was performed. At the control run, only foam and load was placed in the test chamber. The measurement was performed the same way and using the same conditions as for the sample measurements. The control run is then subtracted from the sample run before continued processing of raw data.

TABLE 2

| Sample | PV₅₀ |
|---|---|
| Foam XII | 5.3 cm H₂O |
| Foam XV | 17 cm H₂O |

### EXAMPLE 3

Absorption Capacity

The total absorption capacity per volume unit of the absorbent structure in a dry condition has been estimated for six samples.

Tested Materials

Sample 1 is a mixed structure of chemically manufactured cellulosic fluff pulp from Weyerhauser and a particulate polyacrylate-based super absorbent material from BASF. The mixed structure contains 40 percent by weight of super absorbent material based on the total weight of the sample.

Sample 2 is a fiber structure from Weyerhauser. The fiber structure contains 80 percent by weight cross-linked cellulose and 20 percent by weight of thermo fibers.

Sample 3 is a polyester fiber layer having particulate polyacrylate-based super absorbent material bound to the polyester fiber layer. The percentage of super absorbent particles is 60 percent based on the total weight of the sample.

Sample 4 is a polyacrylate-based super absorbent foam layer. The foam layer is denoted Foam XII and is more closely described in Example 2.

Sample 5 is a polyacrylate-based super absorbent foam layer. The foam layer is denoted Foam XV and is more closely described in Example 2.

Sample 6 is a viscose foam material, i.e., a foam material from regenerated cellulose.

The measurement was performed by first weighing the dry sample. The volume of dry sample is then obtained by dividing the weight of the dry sample with the density of the dry sample. The sample was then saturated using a NaCl solution containing 0.9 percent NaCl by weight. The NaCl solution was delivered to the sample in excess. Thereafter, the saturated sample was weighed. The amount of liquid absorbed was obtained by subtracting the weight of the dry sample from the weight of the saturated sample.

The total absorption capacity, measured in grams per cubic centimeters of the absorbent material in a dry condition, is presented in Table 3.

TABLE 3

| | Absorption capacity (grams liquid/cm³) | | |
|---|---|---|---|
| | ρ = 0.50 | ρ = 0.71 | ρ = 0.91 |
| Sample | g/cm³ | g/cm³ | g/cm³ |
| Sample 1 | 15 | 19 | 22 |
| Sample 2 | 6 | 6 | 8 |
| Sample 3 | 14 | 20 | 26 |
| Sample 4 | 27 | 38 | 49 |
| Sample 5 | 13 | 19 | 24 |
| Sample 6 | 14 | 20 | 26 |

The results clearly show that Foam XII, i.e., sample 4, significantly exhibits a higher total absorption capacity, measured in grams per cubic centimeters of the absorbent material in a dry condition.

1. Absorbent article exhibiting a liquid permeable surface and an opposite at least substantially liquid impermeable surface and comprising an absorbent structure wherein the absorbent structure comprises a super absorbent porous structure exhibiting a Gurley stiffness value being lower than 1000 mg and a density in a dry condition exceeding 0.5 g/cm³.

2. Absorbent article according to claim 1, wherein the super absorbent porous structure is a polyacrylate-based foam.

3. Absorbent article according to the claim 1 wherein the super absorbent porous structure in a dry condition exhibits a density exceeding 0.7 g/cm³.

4. Absorbent article according to claim 1, wherein the super absorbent porous structure exhibits a Gurley stiffness value being lower than 700 mg.

5. Absorbent article according to claim 1, wherein the super absorbent porous structure exhibits a Gurley stiffness value being lower than 500 mg.

6. Absorbent article according to claim 1, wherein the total absorption capacity per cubic centimeter of the super absorbent porous structure in a dry condition is at least 15 g/cm³.

7. Absorbent article according to claim 1, wherein the absorbent structure comprises an acquisition portion and a final storage portion, whereby the super absorbent porous structure constitutes the acquisition portion and that the acquisition portion exhibits a drainage rate, measured by a pore volume distribution device, being such that at least 50% of the drainable pores in the super absorbent porous structure are emptied from liquid at a pressure being lower than 12 cm H₂O.

8. Absorbent article according to claim 1, wherein the acquisition portion exhibits a drainage rate, measured by a pore volume distribution device, being such that at least 50% of the drainable pores in the super absorbent porous structure are emptied from liquid at a pressure being lower than 8 cm H₂O.

9. Absorbent article according to claim 1, wherein the final storage portion at least comprises a first storage layer, wherein the first storage layer is comprised of cellulosic fibers and super absorbent material, wherein the amount of super absorbent material calculated on the total weight of the first storage layer in a dry condition is at least 50% by weight.

10. Absorbent article according to claim 9, wherein the amount of super absorbent material, calculated on the total weight of the first storage layer in a dry condition is at least 70% by weight.

11. Absorbent article according to claim 1, wherein the article is a diaper, an incontinence guard, or a sanitary napkin.
